(19) United States
(12) Patent Application Publication
(10) Pub. No.: US 2026/0272552 A1
(43) Pub. Date: Sep. 17, 2026

(54) ABLATION TARGET IDENTIFICATION VIA MODELING OF ARRHYTHMOGENESIS

(71) Applicant: THE JOHNS HOPKINS UNIVERSITY, Baltimore, MD (US)

(72) Inventors: Natalia A. TRAYANOVA, Baltimore, MD (US); Adityo PRAKOSA, Baltimore, MD (US); Ryan BRADLEY, Baltimore, MD (US); Evgueni KHOLMOVSKI, Baltimore, MD (US); Syed Yusuf ALI, Baltimore, MD (US); Carolyna YAMAMOTO, Baltimore, MD (US); Shane LOEFFLER, Baltimore, MD (US)

(21) Appl. No.: 19/167,444

(22) PCT Filed: Mar. 18, 2024
(86) PCT No.: PCT/US2024/020413

§ 371 Date: Sep. 22, 2025

Related U.S. Application Data
(60) Provisional application No. 63/491,862, filed on Mar. 23, 2023.

(51) Int. Cl. A61B 34/10; A61B 18/00; G06T 5/00; G06T 7/00; G06T 7/11

(52) U.S. Cl. CPC A61B 34/10; A61B 18/00; G06T 5/00; G06T 7/0012; G06T 7/11; A61B 2018/00351; A61B 2018/00577; A61B 2034/105; A61B 2034/107; G06T 2207/10088; G06T 2207/20084; G06T 2207/20112

(57) ABSTRACT

Techniques for providing a cardiac ablation strategy personalized for a patient are provided. The techniques include: obtaining and segmenting a 3D late gadolinium enhancement cardiac MRI of the patient; constructing a patient-specific bi-atrial 3D model that incorporates fibrotic and non-fibrotic tissues; assigning myocardial fiber from a bi-atrial human fiber atlas to the model; automatically distributing pacing locations throughout left and right atria of the model; assigning a PVI lesion in the model; executing a simulation of pacing for each of the pacing locations; identifying locations in atrial substrate of the model capable of sustaining reentrant activity and assigning the locations as ablation targets; connecting each ablation target to a non-conductive anatomical feature or another target; incorporating a corresponding ablation lesion into the model; repeating the executing and the determining to identify emergent locations and assigning the emergent locations as corresponding ablation targets; and providing a representation of an ablation strategy.

### CROSS-REFERENCE TO RELATED APPLICATIONS

This application is the national stage entry of International Patent Application No. PCT/US2024/020413, filed on Mar. 18, 2024, and published as WO 2024/196871 A2 on Sep. 26, 2024, which claims the benefit of U.S. Provisional Patent Application No. 63/491,862, filed on Mar. 23, 2023, which are hereby incorporated by reference herein in their entireties.

### GOVERNMENT SUPPORT

This invention was made with government support under grant NIH HL141074 awarded by the National Institutes of Health. The government has certain rights in the invention.

### FIELD

This disclosure relates generally to the treatment of atrial fibrillation.

### BACKGROUND

Atrial fibrillation (AF) is an emerging global health crisis. About 1-2% of individuals worldwide currently suffer from AF, and its prevalence is expected to increase 2.5-fold in the next 40 years. For symptomatic drug-refractory AF patients, the recommended therapy is catheter-based ablation, which electrically isolates arrhythmia triggers in the pulmonary veins (PVs), a procedure referred to as PVI. However, the success rate of AF catheter ablation is 50-75% in various studies, and is worse in patients with persistent AF, who typically develop fibrosis. Furthermore, supplementary ablative strategies targeting fibrotic substrate in patients undergoing PVI thus far have proven ineffective.

### SUMMARY

According to various embodiments, a method of providing a cardiac ablation strategy personalized for a patient is presented. The method includes: obtaining a 3D late gadolinium enhancement cardiac MRI of the patient; segmenting, automatically and using an artificial neural network, a left and right atrial epicardial surface of the 3D late gadolinium enhanced cardiac MRI of the patient, whereby a segmentation is produced; constructing a patient-specific bi-atrial 3D model from the segmentation, where the model incorporates fibrotic and non-fibrotic tissues; assigning myocardial fiber from a bi-atrial human fiber atlas to the 3D model; distributing automatically a plurality of pacing locations throughout left and right atria of the 3D model; assigning a pulmonary vein isolation (PVI) lesion in the model; executing a simulation of pacing for each of the plurality of pacing locations to test inducibility of reentrant activity; identifying, based on the executing, locations in atrial substrate of the 3D model capable of sustaining reentrant activity and assigning the locations as corresponding ablation targets; connecting each ablation target to a non-conductive anatomical feature or another target; incorporating an ablation lesion corresponding to each ablation target into the 3D model; repeating at least the executing and the determining to identify emergent locations capable of sustaining emergent reentrant activity and assigning the emergent locations as corresponding ablation targets; and providing a representation of an ablation strategy based on the determining, the incorporating, and the repeating.

Various optional features of the above method embodiments include the following. The providing may include exporting the ablation strategy as a file compatible with an electroanatomic mapping system, where the file includes data representing left and atrial surfaces with an ablation lesion marking. The method may include: registering the ablation strategy to the heart of the patient; and ablating the patient's heart according to the ablation strategy. The method may include repeating the executing, the determining, and the incorporating until the model is not inducible for sustained reentrant activity from any pacing site. The method may include calculating personalized image intensity thresholds for left and right atria to define fibrotic tissues. The method may include correcting image intensity inhomogeneity of the 3D late gadolinium enhanced cardiac MRI of the patient due to at least one of: spatially variable sensitivities, of MRI coils or non-uniform RF pulse profile. The method may include performing quality control of the segmentation. The plurality of pacing locations may be distributed uniformly throughout the left and right atria close to the fibrotic tissue. The non-conductive anatomical feature may include a valve or a pulmonary vein isolation lesion. The executing may include performing the simulation of pacing using a Lattice Boltzmann Method.

According to various embodiments, a system for providing a cardiac ablation strategy personalized for a patient, the system including: a non-transitory computer readable medium including instructions; and at least one electronic processor that executes the instructions to perform operations including: obtaining a 3D late gadolinium enhancement cardiac MRI of the patient; segmenting, automatically and using an artificial neural network, a left and right atrial epicardial surface of the 3D late gadolinium enhanced cardiac MRI of the patient, whereby a segmentation is produced; constructing a patient-specific bi-atrial 3D model from the segmentation, where the model incorporates fibrotic and non-fibrotic tissues; assigning myocardial fiber from a bi-atrial human fiber atlas to the 3D model; distributing automatically a plurality of pacing locations throughout left and right atria of the 3D model; assigning a pulmonary vein isolation (PVI) lesion in the model; executing a simulation of pacing for each of the plurality of pacing locations to test inducibility of reentrant activity; identifying, based on the executing, locations in atrial substrate of the 3D model capable of sustaining reentrant activity and assigning the locations as corresponding ablation targets; connecting each ablation target to a non-conductive anatomical feature or another target; incorporating an ablation lesion corresponding to each ablation target into the 3D model; repeating at least the executing and the determining to identify emergent locations capable of sustaining emergent reentrant activity and assigning the emergent locations as corresponding ablation targets; and providing a representation of an ablation strategy based on the determining, the incorporating, and the repeating.

Various optional features of the above system embodiments include the following. The providing may include exporting the ablation strategy as a file compatible with an electroanatomic mapping system, where the file includes data representing left and atrial surfaces with an ablation lesion marking. The operations may further include registering the ablation strategy to the heart of the patient. The operations may further include repeating the executing, the determining, and the incorporating until the model is not inducible for sustained reentrant activity from any pacing site. The operations may further include calculating personalized image intensity thresholds for left and right atria to define fibrotic tissues. The operations may further include correcting image intensity inhomogeneity of the 3D late gadolinium enhanced cardiac MRI of the patient due to at least one of: spatially variable sensitivities, of MRI coils or non-uniform RF pulse profile. The operations may further include performing quality control of the segmentation. The plurality of pacing locations may be distributed uniformly throughout the left and right atria close to the fibrotic tissue. The non-conductive anatomical feature may include a valve or a pulmonary vein isolation lesion. The executing may include performing the simulation of pacing using a Lattice Boltzmann Method.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 is a flow chart for a method of providing a cardiac ablation strategy personalized for a patient, according to various embodiments;

FIG. 2 illustrates a late gadolinium enhancement magnetic resonance image (LGE-MRI) image according to various embodiments;

FIG. 3 illustrates a segmentation of atrial boundaries in an LGE-MRI image, using a neural network, according to various embodiments;

FIG. 4 illustrates quality control applied to a segmented LGE-MRI image according to various embodiments;

FIG. 5 illustrates a patient-specific bi-atrial 3D model constructed from a segmented LGE-MRI image, where the model incorporates fibrotic and non-fibrotic tissues, according to various embodiments;

FIG. 6 illustrates a finite element mesh generated for a patient-specific 3D model, according to various embodiments;

FIG. 7 illustrates a myocardial fiber assignment from a bi-atrial human fiber atlas to a patient-specific 3D model, according to various embodiments;

FIG. 8 illustrates an example pacing location in a patient-specific 3D model, according to various embodiments;

FIG. 9 illustrates an assigned pulmonary vein isolation (PVI) lesion in a patient-specific 3D model, according to various embodiments;

FIG. 10 illustrates identification of locations, in atrial substrate of a patient-specific 3D model, capable of sustaining reentrant activity, according to various embodiments;

FIG. 11 illustrates an assignment of an ablation target in a patient-specific 3D model, according to various embodiments;

FIG. 12 illustrates how pacing from different locations can identify different ablation targets in a patient-specific 3D model, according to various embodiments;

FIG. 13 illustrates virtual ablation of all ablation targets in a patient-specific 3D model, according to various embodiments;

FIG. 14 illustrates connecting ablation targets to a non-conductive anatomical feature in a patient-specific 3D model, according to various embodiments;

FIG. 15 illustrates an identification of an emergent reentrant driver in a patient-specific 3D model, according to various embodiments;

FIG. 16 illustrates incorporation of an ablation lesion corresponding to an emergent reentrant driver into a patient-specific 3D model, according to various embodiments; and

FIG. 17 illustrates registering a representation of an ablation strategy with a patient, according to various embodiments.

### DESCRIPTION OF THE EXAMPLES

Reference will now be made in detail to example implementations, illustrated in the accompanying drawings. These examples are described in sufficient detail to enable those skilled in the art to practice the invention and it is to be understood that other examples may be utilized and that changes may be made without departing from the scope of the invention. The following description is, therefore, merely exemplary.

Some embodiments provide a personalized ablation approach that targets remodeled bi-atrial substrate and thereby improves the efficacy of therapy and eliminates repeated procedures. Some embodiments provide a personalized prediction of optimal extra-PVI bi-atrial ablation targets that render, with minimum lesions size, the atrial substrate not inducible for AF from any stimulus location. Some embodiments employ an iterative approach for identifying a personalized bi-atrial substrate ablation strategy that accounts for the sequential change in atrial substrate after each ablation round (PVI, then any extra-PVI targets). The personalized set of optimal ablation targets may be implemented in a clinical workflow and used to guide the ablation procedure in patients with persistent AF and atrial fibrosis. Some embodiments are expected to result in dramatically improved treatment efficacy and reduce the need for repeated ablations.

The inventors conducted a study that used an embodiment to perform ablation on 26 patients with persistent AF, with two-year follow-ups. For persistent AF, the outcome of ablation using the current state of the art is about 50% (i.e., it fails about 50% of the cases). The preliminary results from the study using the embodiment showed a two-year success rate of 76%, a significant improvement over the prior art. Furthermore, many patients after atrial fibrillation ablation experience a simpler form of arrhythmia, called atrial tachycardia (AT), which requires an additional procedure. AT is quite frequent in these patients. The preliminary results of the study using the embodiment showed a very high success rate in preventing AT, at 96%.

These and other features and advantages are shown and described herein in reference to the figures.

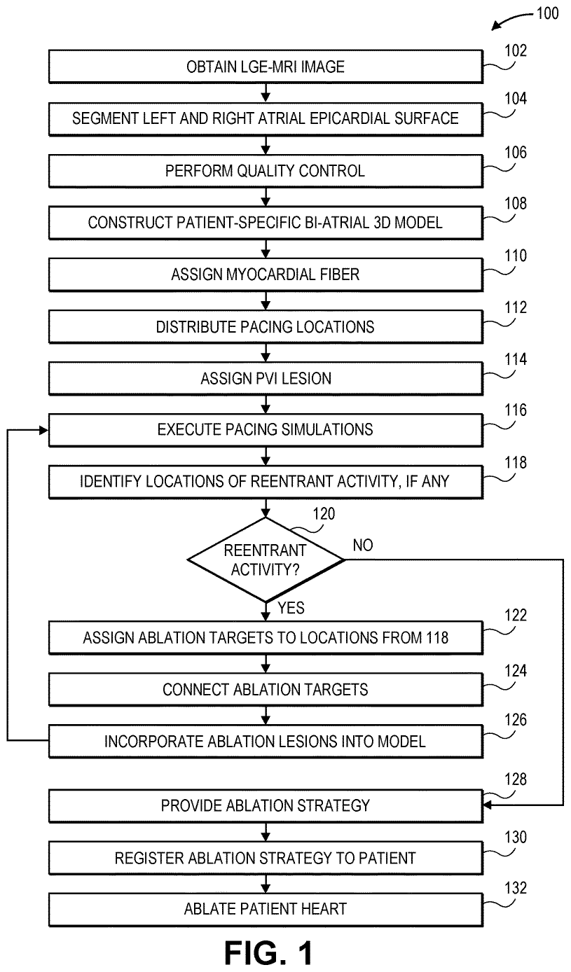

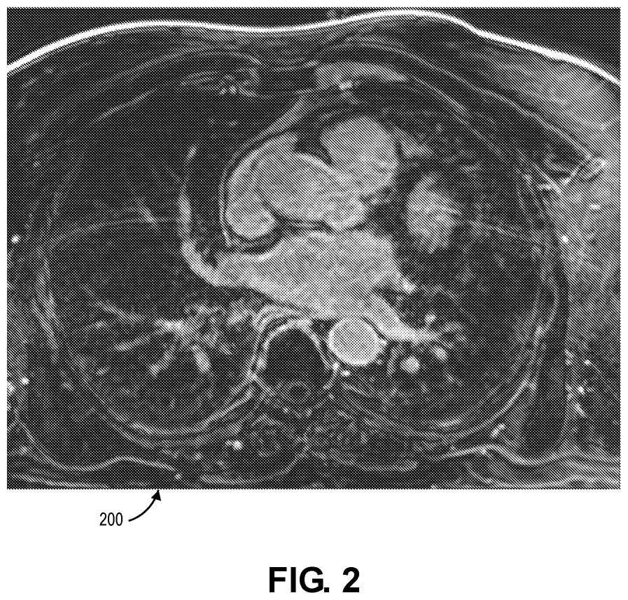

FIG. 1 is a flow chart for a method 100 of providing a cardiac ablation strategy personalized for a patient, according to various embodiments. Note that various embodiments may include any sub-combination of elements shown in FIG. 1. The method 100 is described throughout the remainder of this disclosure in reference to FIGS. 2-17. The method 100 may be conceptualized as including four main components, as follows:
(i) Personalized bi-atrial model construction from LGE-MRI and other data (e.g., FIG. 1, references 102, 104, 106, 108, 110),
(ii) Personalized simulation of substrate AF inducibility (e.g., FIG. 1, references 112, 116, 118),
(iii) Iterative personalized extra-PVI ablation strategy to determine all extra-PVI targets, rendering the substrate non-inducible for AF (e.g., FIG. 1, references 120, 122, 124, 126, 128), and
(iv) Implementation in the clinical workflow (e.g., FIG. 1, references 130, 132).

At 102, the method 100 includes obtaining an LGE-MRI image of a patient. The LGE-MRI image may be obtained using conventional techniques. The LGE-MRI image may be obtained directly from an MRI scanner, through retrieval from electronic storage, or via a different technique. FIG. 2 illustrates an LGE-MRI image 200 according to various embodiments.

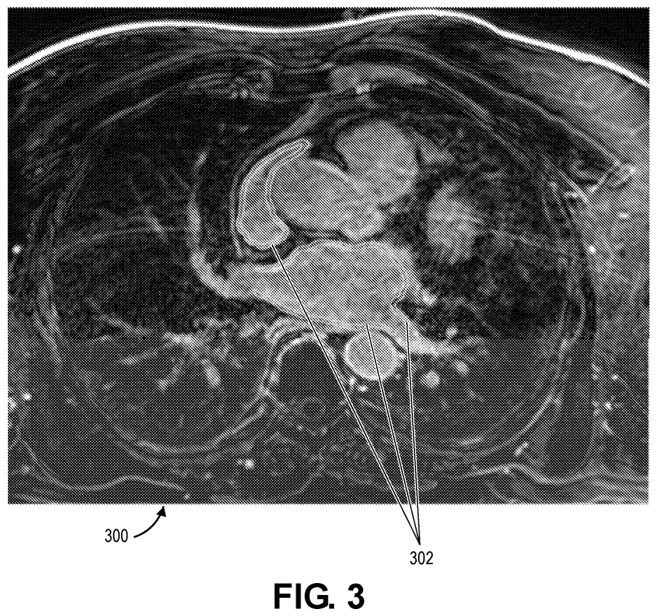

At 104, the method 100 includes segmenting left and right atrial epicardial surfaces. The segmentation may be performed using a deep learning approach to bi-atrial geometry and fibrosis segmentation. By way of non-limiting example, and according to a reduction to practice, the deep learning approach may include a cascade of (e.g., four) artificial neural networks (NNs). Thus, at 104, the method may include performing automatic segmentation of left and right atrial epicardial surface on a 3D LGE-MRI using a neural network. FIG. 3 illustrates a segmentation 302 of atrial boundaries in an LGE-MRI image 300, using a neural network, according to various embodiments.

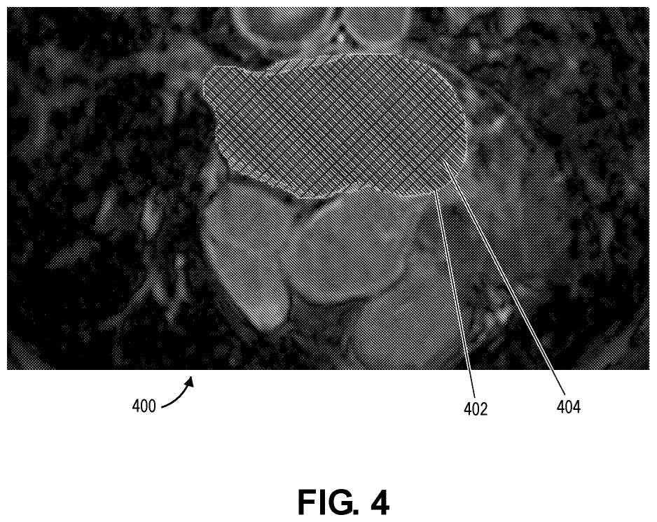

At 106, the method 100 includes performing quality control on the segmentation. The quality control may include any, or a combination of quality control techniques. According to some embodiments, the actions of 106 includes correcting any 3D MRI-LGE image intensity inhomogeneity, e.g., due to spatially variable sensitivities of MRI coils, non-uniform RF pulse profile, etc. According to some embodiments, the actions of 106 may include determining personalized image intensity thresholds for the left and right atria to define fibrotic tissues. The personalized image intensity thresholds may utilize a ratio for fibrosis detection based on the observation that other anatomical structures with high fibrosis are enhanced in atrial LGE-MRI and thus can be used as a personalized reference. According to some embodiments, the actions of 106 may include performing quality control of the segmentation. FIG. 4 illustrates quality control 400 applied to a segmented LGE-MRI image according to various embodiments. As shown in FIG. 4, the quality control includes manually performing minor corrections to a single MRI image slice. As illustrated in FIG. 4, the automatic segmentation boundary 404 is manually corrected to the revised boundary 402.

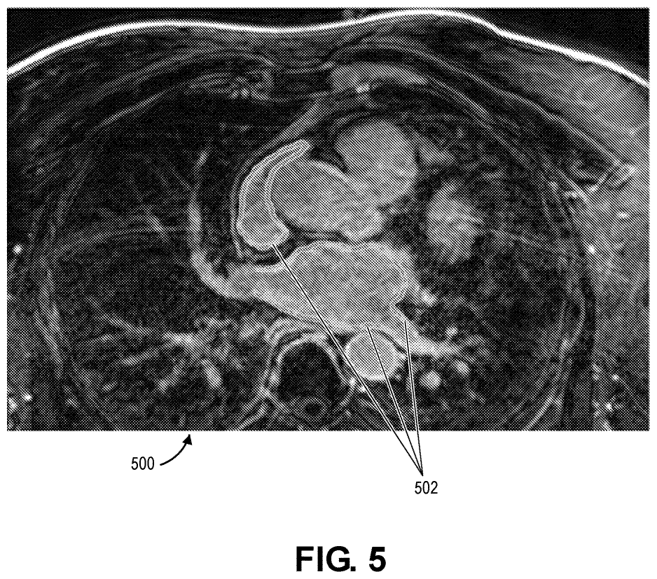

At 108, the method 100 includes constructing a patient-specific bi-atrial 3D model from the segmentation, incorporating fibrotic and non-fibrotic tissues. FIG. 5 illustrates a patient-specific bi-atrial 3D model 500 constructed from a segmented LGE-MRI image, where the model incorporates fibrotic and non-fibrotic tissues 502, according to various embodiments.

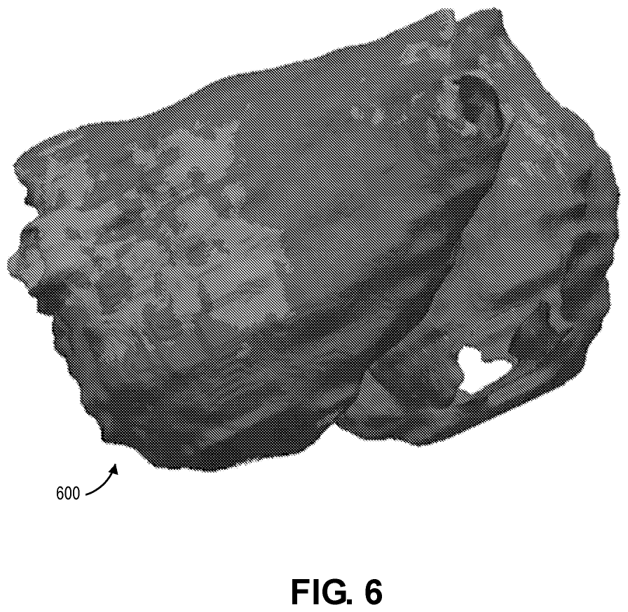

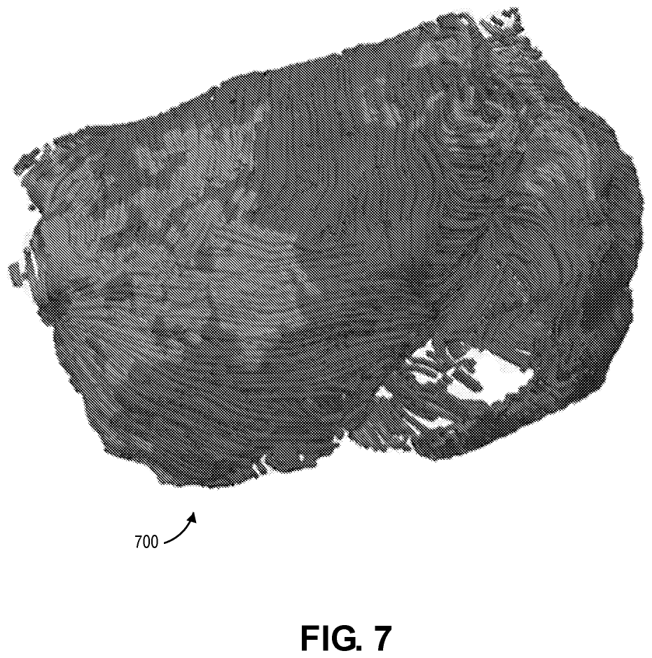

At 110, the method 100 includes assigning myocardial fiber to a patient-specific bi-atrial 3D model. The fiber assignment may be preceded by generating a finite element mesh to the patient-specific 3D model, and the fiber assignment may utilize the finite element mesh. FIG. 6 illustrates an example finite element mesh 600 generated for a patient-specific 3D model, according to various embodiments. According to some embodiments, the myocardial fiber may be assigned from a bi-atrial human fiber atlas to the patient-specific 3D model. FIG. 7 illustrates a myocardial fiber assignment 700 from a bi-atrial human fiber atlas to a patient-specific 3D model, according to various embodiments.

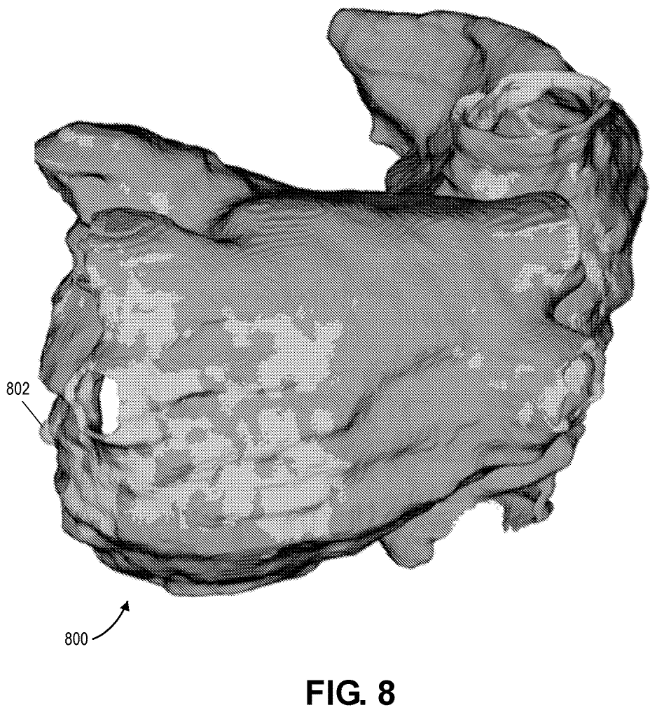

At 112, the method 100 includes automatically distributing pacing locations throughout left and right atria of the patient-specific 3D model. By way of non-limiting example, 40 pacing locations may be automatically distributed in a uniform manner throughout left and right atria. The pacing locations may be distributed in proximity to fibrotic tissue and/or may be uniformly distributed, according to various embodiments. FIG. 8 illustrates an example such pacing location 802 in a patient-specific 3D model 800, according to various embodiments.

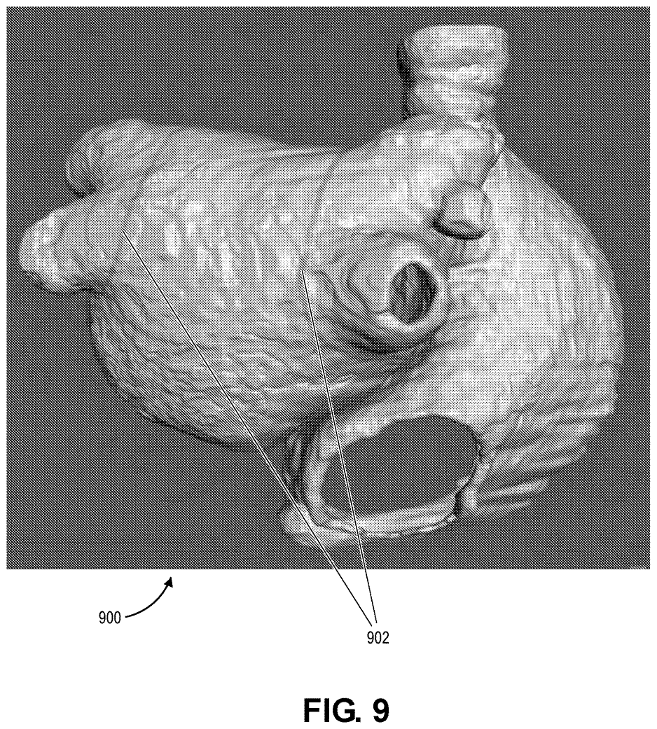

At 114, the method 100 includes assigning a PVI lesion in the model. The PVI lesion may be modeled according to a standard of care, for example. The PVI lesion, as well as any other ablation lesions referenced herein, may be represented as non-conductive regions in the patient-specific 3D model. FIG. 9 illustrates an assigned PVI lesion 902 in a patient-specific 3D model 900, according to various embodiments.

After the actions of 114, the method 100 proceeds, within the patient-specific 3D model, to iteratively: probe for extra-PVI substrate rotor inducibility by pacing from numerous sites, determine, from the pacing, locations capable of sustaining persistent drivers, and ablate the determined locations. Each ablation is followed by an inducibility test, and this sequence is repeated until full substrate non-inducibility from any pacing site is achieved. This iterative approach is shown and described in reference to 116, 118, 120, 122, 124, and 126 presently.

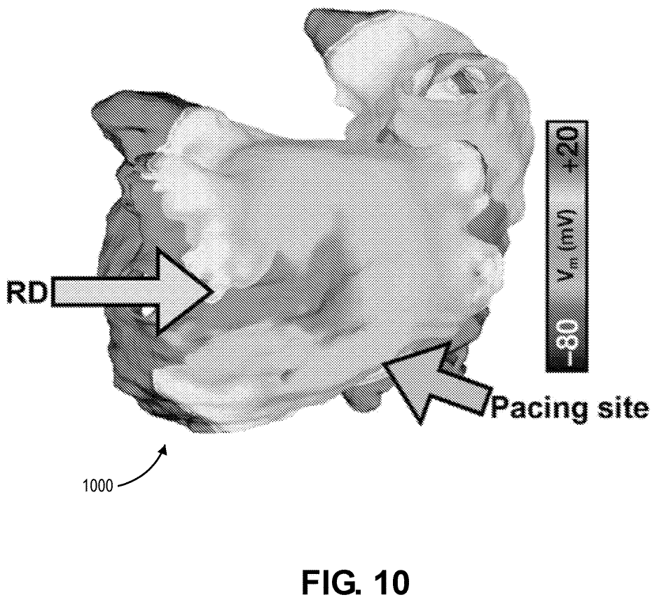

At 116, the method 100 includes executing, using the patient-specific 3D model, a simulation of pacing at the pacing locations of 112 to test substrate rotor inducibility of reentrant activity. The actions of 116 may include, in parallel, running the simulations of pacing from the pacing locations to assess inducibility of reentrant activity in the substrate. That is, because each simulation of pacing at a given location and subsequent wave propagation is independent of any other such simulation at another location, multiple (e.g., all) such simulations can be executed together in parallel. FIG. 10 illustrates an identification of a pacing site (PS) in atrial substrate of a patient-specific 3D model 1000, according to various embodiments.

According to some embodiments, the simulation of pacing may be performed using a finite element method, which solves a system of ordinary differential equations (representing cellular electrical signal generation) together with partial differential equations (representing the spread of this signal through the heart, based on a continuum finite element approach).

According to some embodiments, the simulation of pacing may be performed using the Lattice Boltzmann Method (LBM), e.g., as disclosed in J. O. Campos, R. S. Oliveira, R. W. dos Santos, B. M. Rocha, Lattice Boltzmann method for parallel simulations of cardiac electrophysiology using GPUs, J. Comp. and Appl. Math., v. 295, 2016, pp. 70-82. In general, pacing provides a stimulus that leads to an emergent electrical wave, and the simulation may include simulating a delivery of the stimulus and simulating the subsequent electrical wave propagation through the heart. Embodiments that utilize LBM improve upon embodiments that utilize a finite element method that numerically solves differential equations. For example, embodiments that utilize LBM may solve for the signal at the cell level, and the LBM technique may represent the spread of that signal through the heart (like particle flow) without the need to solve partial differential equations. Embodiments that utilize LBM at 116 may achieve a 500-fold speedup in comparison to embodiments that utilize a finite element method.

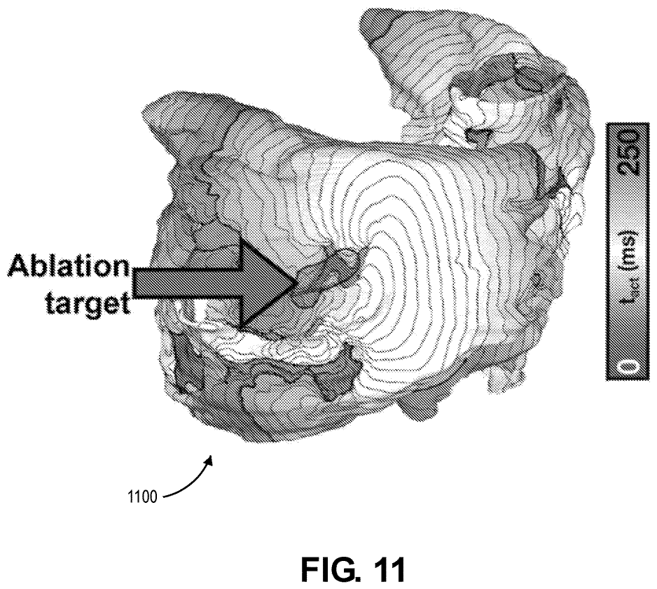

At 118, the method 100 includes identifying, based on 116, locations in the atrial substrate of the patient-specific 3D model capable of sustaining reentrant activity, if any. That is, locations in the virtual PVI and fibrosis substrate of the patient-specific 3D model that are capable of sustaining reentrant activity are identified at 118. These constitute the initial ablation targets. An example reentrant driver (RD) is shown in FIG. 10. In particular, as shown in FIG. 10, pacing at the pacing site (PS) induces a corresponding reentrant driver (RD). The reentrant driver (RD) of FIG. 10 is identified as an ablation target in the patient-specific 3D model 1100, shown in FIG. 11.

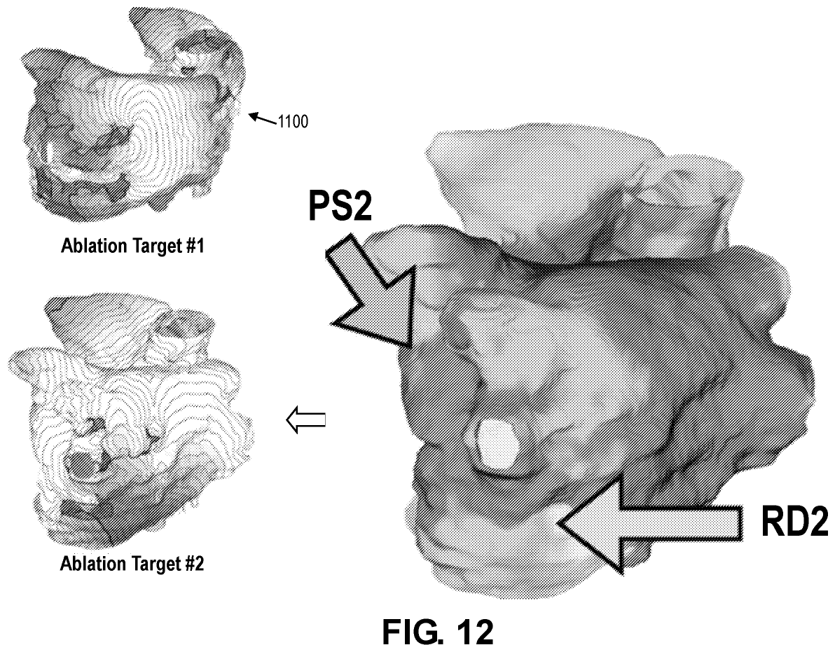

Note that pacing at different locations can identify different reentry drivers and therefore different ablation targets. For example, FIG. 12 illustrates how pacing from different locations can identify different ablation targets as shown in the patient-specific 3D models 1100, 1202, according to various embodiments. In particular, pacing at the pacing site (PS2) induces a corresponding reentrant driver (RD2). The reentrant driver (RD2) is identified as Ablation Target #2, shown at 1202. Note that Ablation Target #2, which is identified by pacing at (PS2), is different from Ablation Target #1, which is identified using pacing at (PS1) (see FIG. 11).

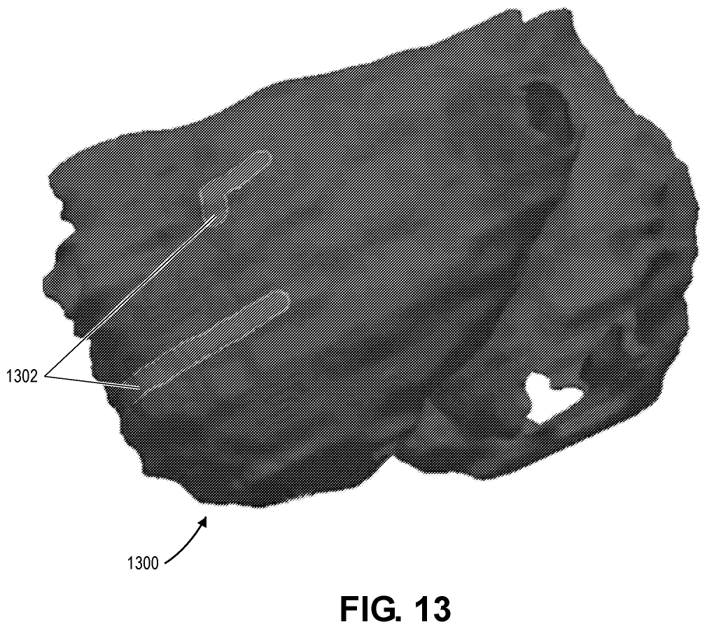

If, at 120, any reentrant activity remains as identified at 116, then the method 100 continues to 122, where ablation targets are assigned as shown and described herein in reference to FIGS. 11 and 12. FIG. 13 illustrates virtual ablation targets 1302 at locations of all reentrant drivers in a patient-specific 3D model 1300, according to various embodiments. The virtual ablation targets 1302 shown in FIG. 13 includes ablation at all ablation sites identified during one iteration round.

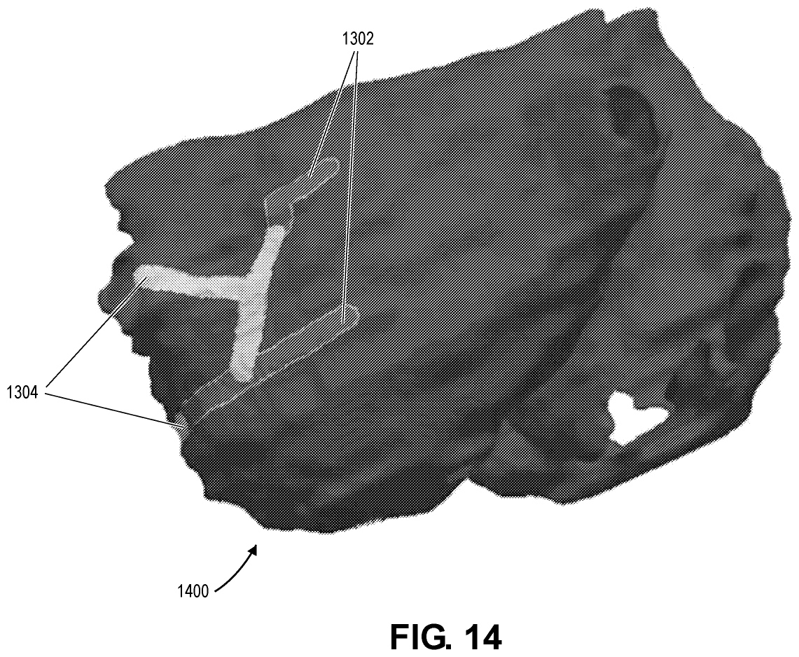

At 124, the method 100 includes connecting the ablation targets to the nearest non-conductive anatomical feature, such as a valve, or to the PVI or another lesion. FIG. 14 illustrates connecting ablation targets 1302 to a non-conductive anatomical feature 1400 in a patient-specific 3D model 1300, according to various embodiments. In particular, FIG. 14 shows connecting the reentrant driver ablation targets 1302 to vessel boundaries (veins or valves) via linear ablation lesions 1304.

At 126, the method 100 includes incorporating the ablation lesions of the preceding actions into the patient-specific 3D model. The ablation lesions may be represented as non-conductive regions in the patient-specific 3D model. After 126, control reverts to 116.

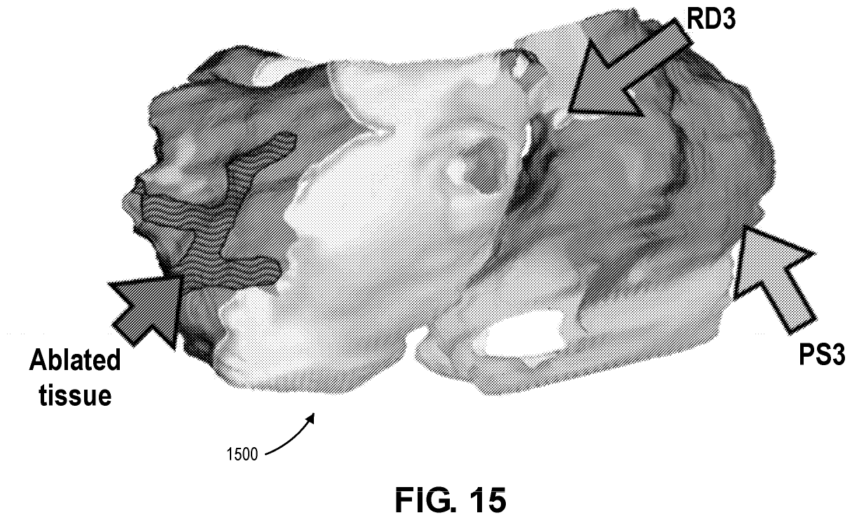

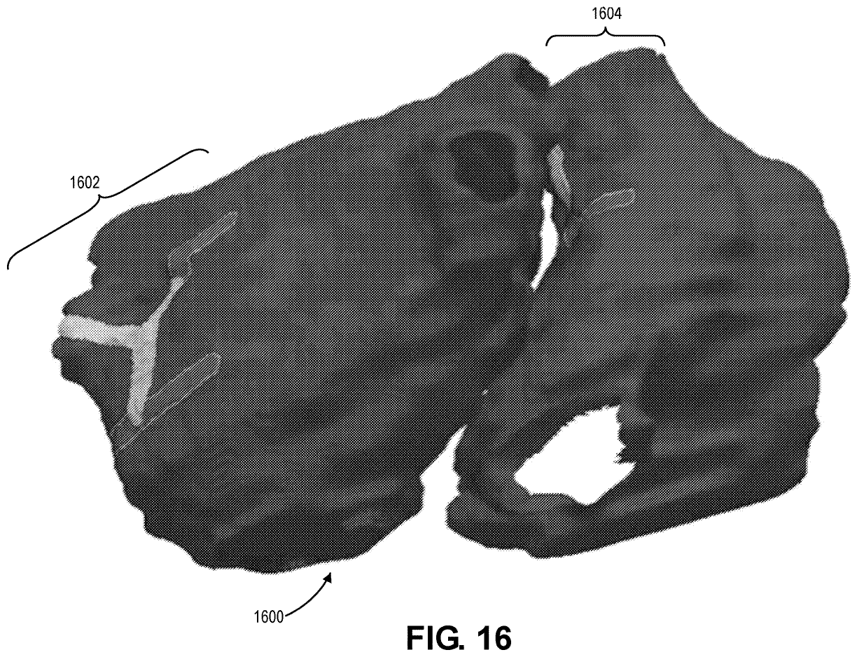

Thus, per the iteration described herein, after incorporating the initial ablation lesions in the patient-specific 3D model (at 114), the pacing from each pacing location (at 116) is repeated to test whether there are any new (emergent) locations capable of sustaining reentrant activity (at 118) in the virtual PVI+lesions+fibrosis substrate. FIG. 15 illustrates an identification of an emergent reentrant driver (RD3) in a patient-specific 3D model 1500, according to various embodiments. In particular, FIG. 15 shows simulated ablated tissue corresponding to the virtual ablation lesion of FIG. 14, together with an emergent reentrant driver (RD3) identified from pacing at the pacing site (PS3). Per the iteration, such emergent targets are then (virtually) ablated in the model (at 122, 126, and 126). FIG. 16 illustrates a patient-specific 3D model 1600 with a virtual ablation lesion 1604 corresponding to the emergent reentrant driver of FIG. 15, together with the previous virtual ablation lesion 1602 shown and described in reference to FIG. 14, according to various embodiments. The iteration may be repeated until the model is not inducible for reentrant activity from any pacing site, and additional ablation lesions may be added to the patient-specific 3D model 1600.

In the absence of reentrant activity, at 120, control passes to 128. At 128, the method 100 includes providing the ablation strategy developed according to the preceding actions. The ablation strategy may be provided to a clinical record system, e.g., an electro anatomical mapping (EAM) system. The ablation strategy may be provided as one or more files stored in persistent memory, over a network connection, or according to a different provision technique. The file or files may include the left and atrial surface with virtual ablation lesion markings. In general, the ablation strategy may be provided in the form of the patient-specific 3D model or a derivation thereof, e.g., a surface with ablation site annotations.

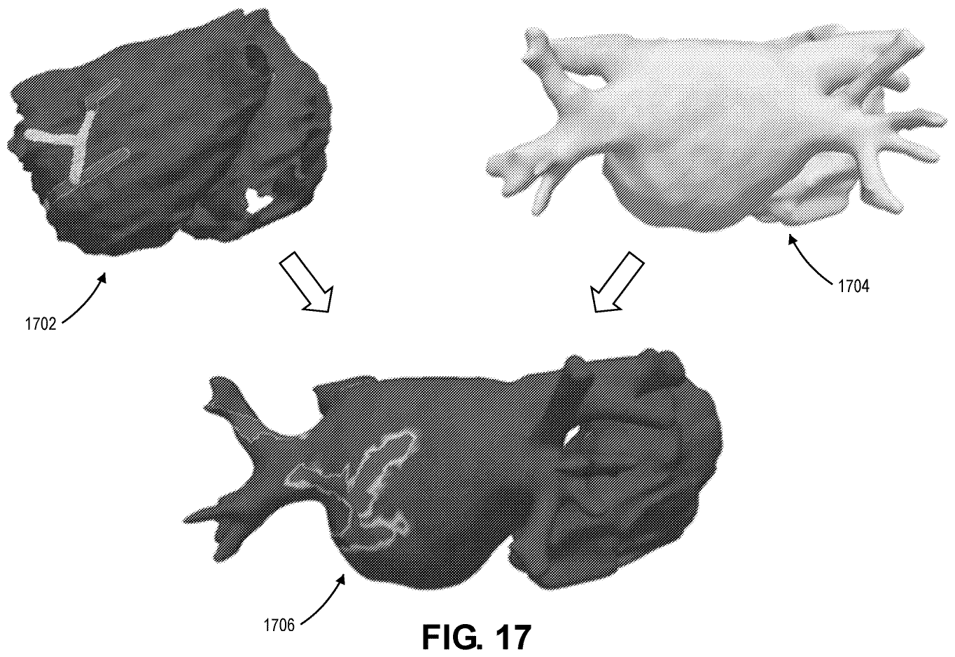

At 130, the method 100 includes registering the ablation strategy with a patient. FIG. 17 illustrates registering a representation of an ablation strategy 1702 with a patient, according to various embodiments. As shown in FIG. 17, the representation of the ablation strategy 1702 may be registered to a representation of atrial geometry 1704 to obtain a registration 1706, which may be in the form of one or more EAM files. The registration 1706 may be used to guide a clinical AF ablation procedure. Either or both of the representation of the ablation strategy 1702 and the registration 1706 may be stored in an EAM system prior to or during a clinical ablation procedure.

At 132, the method 100 includes clinically performing an AF ablation on the patient according to the ablation strategy. The ablation may use any clinical AF ablation technique, such as radiofrequency ablation.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

Embodiments can be implemented using computer readable program instructions that are executed by a processor. These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of providing a cardiac ablation strategy personalized for a patient, the method comprising:
obtaining a 3D late gadolinium enhancement cardiac MRI of the patient;
segmenting, automatically and using an artificial neural network, a left and right atrial epicardial surface of the 3D late gadolinium enhanced cardiac MRI of the patient, whereby a segmentation is produced;
constructing a patient-specific bi-atrial 3D model from the segmentation, wherein the model incorporates fibrotic and non-fibrotic tissues;
assigning myocardial fiber from a bi-atrial human fiber atlas to the 3D model;
distributing automatically a plurality of pacing locations throughout left and right atria of the 3D model;
assigning a pulmonary vein isolation (PVI) lesion in the model;
executing a simulation of pacing for each of the plurality of pacing locations to test inducibility of reentrant activity;
identifying, based on the executing, locations in atrial substrate of the 3D model capable of sustaining reentrant activity and assigning the locations as corresponding ablation targets;
connecting each ablation target to a non-conductive anatomical feature or another target;
incorporating an ablation lesion corresponding to each ablation target into the 3D model;
repeating at least the executing and the determining to identify emergent locations capable of sustaining emergent reentrant activity and assigning the emergent locations as corresponding ablation targets; and
providing a representation of an ablation strategy based on the determining, the incorporating, and the repeating.

2. The method of claim 1, wherein the providing comprises exporting the ablation strategy as a file compatible with an electroanatomic mapping system, wherein the file comprises data representing left and atrial surfaces with an ablation lesion marking.

3. The method of claim 2, further comprising:
registering the ablation strategy to the heart of the patient; and
ablating the patient's heart according to the ablation strategy.

4. The method of claim 1, further comprising repeating the executing, the determining, and the incorporating until the model is not inducible for sustained reentrant activity from any pacing site.

5. The method of claim 1, further comprising calculating personalized image intensity thresholds for left and right atria to define fibrotic tissues.

6. The method of claim 1, further comprising correcting image intensity inhomogeneity of the 3D late gadolinium enhanced cardiac MRI of the patient due to at least one of: spatially variable sensitivities, of MRI coils or non-uniform RF pulse profile.

7. The method of claim 1, further comprising performing quality control of the segmentation.

8. The method of claim 1, wherein the plurality of pacing locations are distributed uniformly throughout the left and right atria close to the fibrotic tissue.

9. The method of claim 1, wherein the non-conductive anatomical feature comprises a valve or a pulmonary vein isolation lesion.

10. The method of claim 1, wherein the executing comprises performing the simulation of pacing using a Lattice Boltzmann Method.

11. A system for providing a cardiac ablation strategy personalized for a patient, the system comprising: a non-transitory computer readable medium comprising instructions; and at least one electronic processor that executes the instructions to perform operations comprising:
obtaining a 3D late gadolinium enhancement cardiac MRI of the patient;
segmenting, automatically and using an artificial neural network, a left and right atrial epicardial surface of the 3D late gadolinium enhanced cardiac MRI of the patient, whereby a segmentation is produced;
constructing a patient-specific bi-atrial 3D model from the segmentation, wherein the model incorporates fibrotic and non-fibrotic tissues;
assigning myocardial fiber from a bi-atrial human fiber atlas to the 3D model;
distributing automatically a plurality of pacing locations throughout left and right atria of the 3D model;
assigning a pulmonary vein isolation (PVI) lesion in the model;
executing a simulation of pacing for each of the plurality of pacing locations to test inducibility of reentrant activity;
identifying, based on the executing, locations in atrial substrate of the 3D model capable of sustaining reentrant activity and assigning the locations as corresponding ablation targets;
connecting each ablation target to a non-conductive anatomical feature or another target;
incorporating an ablation lesion corresponding to each ablation target into the 3D model;
repeating at least the executing and the determining to identify emergent locations capable of sustaining emergent reentrant activity and assigning the emergent locations as corresponding ablation targets; and
providing a representation of an ablation strategy based on the determining, the incorporating, and the repeating.

12. The system of claim 11, wherein the providing comprises exporting the ablation strategy as a file compatible with an electroanatomic mapping system, wherein the file comprises data representing left and atrial surfaces with an ablation lesion marking.

13. The system of claim 12, wherein the operations further comprise:
registering the ablation strategy to the heart of the patient.

14. The system of claim 11, wherein the operations further comprise repeating the executing, the determining, and the incorporating until the model is not inducible for sustained reentrant activity from any pacing site.

15. The system of claim 11, wherein the operations further comprise calculating personalized image intensity thresholds for left and right atria to define fibrotic tissues.

16. The system of claim 11, wherein the operations further comprise correcting image intensity inhomogeneity of the 3D late gadolinium enhanced cardiac MRI of the patient due to at least one of: spatially variable sensitivities, of MRI coils or non-uniform RF pulse profile.

17. The system of claim 11, wherein the operations further comprise performing quality control of the segmentation.

18. The system of claim 11, wherein the plurality of pacing locations are distributed uniformly throughout the left and right atria close to the fibrotic tissue.

19. The system of claim 11, wherein the non-conductive anatomical feature comprises a valve or a pulmonary vein isolation lesion.

20. The system of claim 11, wherein the executing comprises performing the simulation of pacing using a Lattice Boltzmann Method.
